(19) United States
(12) Patent Application Publication
(10) Pub. No.: US 2017/0311803 A1
(43) Pub. Date: Nov. 2, 2017

(54) METHODS, COMPUTER-READABLE MEDIA, AND SYSTEMS FOR MEASURING BRAIN ACTIVITY

(71) Applicant: Yale University, New Haven, CT (US)

(72) Inventor: Joy Hirsch, New York, NY (US)

(73) Assignee: Yale University, New Haven, CT (US)

(21) Appl. No.: 15/520,547

(22) PCT Filed: Nov. 3, 2015
(86) PCT No.: PCT/US15/58835

§ 371 Date: Apr. 20, 2017

Related U.S. Application Data
(60) Provisional application No. 62/075,224, filed on Nov. 4, 2014.

(51) Int. Cl. A61B 5/00; A61B 5/16

(52) U.S. Cl. CPC A61B 5/0042; A61B 5/7246; A61B 5/165; A61B 5/168; A61B 5/4064; A61B 5/4836; A61B 5/726; A61B 5/0075; A61B 5/6803; A61B 5/4848; A61B 2503/04; A61B 2503/06; A61B 2562/0238; A61B 2562/046; A61B 2576/026

(57) ABSTRACT

One aspect of the invention provides a method including: obtaining neural activity data for a first subject and a second subject during verbal interaction between the first subject and the second subject; and calculating coherence between the neural activity data for the first subject and the neural activity data for the second subject. Another aspect of the invention provides a non-transitory, tangible computer-readable medium comprising computer-readable program instructions for implementing the one or more of the methods described herein.

### CROSS-REFERENCE TO RELATED APPLICATION

This application claims priority to U.S. Provisional Patent Application Serial No. 62/075,224, filed Nov. 4, 2014. The entire content of this application is hereby incorporated by reference herein.

### BACKGROUND OF THE INVENTION

Although autism is a developmental disorder with profound disabilities in communication skills, little is known about the brain organization that underlies these early communication skills and their development. Autism Spectrum Disorders (ASDs) are estimated to affect 1 in 88 children. A core of common features is observed in autistic subjects including profound communication and social disability, and repetitive and restricted behaviors and interests. Autism is a developmental disorder usually diagnosed between 2 to 5 years of age, when clear and multiple symptoms are observed.

### SUMMARY OF THE INVENTION

One aspect of the invention provides a method including: obtaining neural activity data for a first subject and a second subject during verbal interaction between the first subject and the second subject; and calculating coherence between the neural activity data for the first subject and the neural activity data for the second subject.

This aspect of the invention can have a variety of embodiments. The neural activity data can be functional near-infrared spectroscopy (fNIRS) signals.

The verbal interaction can include an object naming and narrative task. The verbal interaction can include alternating monologues. The verbal interaction can include dialogue between the first subject and the second subject.

Coherence can be calculated using a cross-correlation algorithm. Coherence can be calculated using a phase-locked wavelet coherence algorithm.

The first subject can be an adult and the second subject is selected from the group consisting of an infant, a toddler, and a child.

The method can further include correlating diminished coherence values with affliction with a social disorder. The social disorder can be a developmental social disorder. The social disorder can be autism. The social disorder can be selected from the group consisting of: schizophrenia, post-traumatic stress disorder, and depression.

Another aspect of the invention provides a non-transitory, tangible computer-readable medium comprising computer-readable program instructions for implementing the one or more of the methods described herein.

Another aspect of the invention provides a system including: at least two NIRS caps, each of NIRS cap including a plurality of optodes; an fNIRS system communicatively coupled to the at least two NIRS caps, the fNIRS system programmed to obtain fNIRS data from the at least two NIRS caps when the at least two NIRS caps are worn by subjects; and a computing device. The computing device is programmed to: obtain fNIRS data for a first subject and a second subject during verbal interaction between the first subject and the subject; calculate coherence between the neural activity data for the first subject and the neural activity data for the second subject.

This aspect of the invention can have a variety of embodiments. The computing device can be further programmed to correlate diminished coherence values with affliction with a social disorder.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1A depicts a system for identifying an individual afflicted with a social disorder according to an embodiment of the invention.

FIG. 1B depicts the use of portions of a system by a mother-infant dyad during interpersonal interaction according to an embodiment of the invention.

FIG. 2 depicts a system for identifying an individual afflicted with a social disorder according to an embodiment of the invention.

FIGS. 3A-3D depict testing protocols according to embodiments of the invention.

FIG. 4 depicts fNIRS signals from region of region of interest (the dorsolateral prefrontal cortex) exhibiting increased correlation during dialogue (red) in comparison to monologue (blue). Panels (i) and (ii) depicts the raw signal amplitude. In panel (i), subject 1 (S1, depicted in solid line) and subject 2 (S2, depicted in broken line) are anticorrelated as they alternate takes. In panels (ii) and (iii), increased group amplitude is seen during dialogue. Panels (iv) and (v) depict correlation for continuous residual signals for each subject. As seen in panels (iv) and (v), correlation between S1 and S2 is greater for dialogue. Panel (vi) depicts increased group correlation during dialogue.

FIG. 5 depicts the measurement of the blood oxygenation level-dependent (BOLD) signal with fNIRS. The left panel depicts the position of the 52 channels in a standard brain volume. The right panel depicts positive BOLD signals in one channel activated by passive listening to language.

FIG. 6 depicts the optode placement and locations of fNIRS channels. The use of 10 emitters and 10 recorders results in 30 channels in one hemisphere of each brain placed in homologous locations with a spatial resolution of approximately 3 cm. The upper left panel of the figure below shows an example an optode array with both detectors (blue units) and emitters (read units). The upper right panel shows a typical matrix superimposed on a standard brain. Channels that are assumed to be sources of these signals are located in between the matrix of emitters and detectors and are spaced at approximately 3 cm. The bottom row of panels show these channels identified by yellow numbers interspersed between the optical matrix.

FIG. 7 depicts absorption spectra for deoxyhemoglobin and oxyhemoglobin. The functions illustrate a maximum absorption difference for Oxy-Hb and Deoxy-Hb at 780 nm and 830 nm. The oxygen concentration in the blood affects the wavelengths that are reflected. The Modified Beer-Lambert Law can be used to convert direct measurements of light attenuation at the three wavelengths (780 nm, 805 nm and 830 nm) into corresponding changes for substances of interest: deoxyhemoglobin, oxyhemoglobin, and total hemoglobin.

FIG. 8 depicts data from preliminary two-brain studies of dialogue vs. monologue. The left panels depict the residual signals for subjects S1 and S2, which are the original fNIRS BOLD signal minus only the periodic component of the hemodynamic response function expected based on the experimental time series. The right panels depicts the coherence between the residual signals in the dorsolateral prefrontal cortex (DLPFC) for subjects 1 and 2 represented along the time course of the experiment (x-axis) according to the color scale. Hot colors represent high correlation and cool colors represent low correlations. The y-axis indicates the integration time span (frequency of the components), where 10 seconds indicates the lowest frequency which increases as the axis ascends. Note that the communicating subjects (dialogue condition) demonstrate periodic synchrony (bright colored vertical patches) during the dialogue condition that is not present during the monologue condition. These findings are consistent with interaction-induced cross-brain synchrony at the DLPFC not dependent upon the periodicity of the listening and talking epochs. Both wavelet analysis and cross-correlation analysis of the residual signal converge to provide the first direct evidence for brain-to-brain synchrony during interpersonal interaction.

FIG. 9 depicts results of coherence analysis of cross-brain synchronization.

FIG. 10 depicts results of coherence analysis between the DLPFC and fusiform gyms. Coherence between the DLPFC and fusiform gyms was greater for the face-to-face condition than for the occluded condition at epoch length (wavelet) 4.22 sec, p<0.001. This finding was bilateral across pairs of subjects and unbiased with respect to regions observed.

FIG. 11 depicts results of coherence analysis between the DLPFC and Broca's area. Coherence between the DLPFC and Broca's area was greater for the face-to-face condition than for the occluded condition at epoch length (wavelet) 8.42 sec, p<0.001. This finding was bilateral across pairs of subjects and unbiased with respect to regions observed.

FIG. 12 depicts an experimental setup in which two subjects each wearing FNIRS caps and eye tracking glasses face each other according to an embodiment of the invention.

FIGS. 13A and 13B depict the gaze criterion for eye-to-eye and eye-to-picture conditions according to an embodiment of the invention.

FIG. 14 depicts eye-gaze block paradigm and behavior according to an embodiment of the invention.

FIG. 15 depicts eye gaze behavior for eye-to-eye and eye-to-picture conditions according to embodiments of the invention.

FIG. 16 depicts raw fNIRS signals for a single subject, single channel, and single location according to an embodiment of the invention.

FIGS. 17A and 17B depict channel location for the gaze experiments described herein.

FIGS. 18A and 18B depict contrast results for eye-to-eye conditions relative to eye-to-picture conditions.

FIG. 19 depicts an approach to identifying cross-brain coherence according to an embodiment of the invention. FIG. 20 depicts a comparison of cross-brain coherence according to an embodiment of the invention. Red lines and shading represent coherence during eye-to-eye conditions. Blue lines and shading represent coherence during eye-to-picture conditions.

FIG. 21 depicts the components of cross-brain coherence according to an embodiment of the invention.

FIG. 22 depicts the cross-brain coherence across brains during dialogue when the subjects are in agreement.

FIG. 23 depicts the cross-brain coherence across brains during dialogue when the subjects are in disagreement.

FIG. 24 depicts the single brain results for disagreement relative to agreement.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Autism is often expected suspected by mothers or grandmothers because the infant or young child does not look at the mother. However, there is currently no way to objectively quantify or understand this interpersonal interaction.

Although abnormal behaviors (e.g., diminished eye contact, delays on babbling and gesturing, diminished engagement of face-to-face interactions) can be observed during early infancy, several recent behavioral studies on infants at risk for autism have demonstrated that during the first months of life there are no clear differences in cognitive and language development between low risk controls (infants without family history of ASD) and high risk infants (babies with ASD siblings) that later meet criteria for ASD.

Aspects of the invention utilize synchronicity of language and social brain areas of autistic children during real-life, interactive communication with their mothers (or mother surrogates). Without being bound by theory, Applicant expects decreased cross-brain synchronization of language and associated regions during mother's speech in autistic children is expected compared to typically developing children. On the other hand, Applicant expects similar cross-brain synchronization of language regions during songs. The cross-brain synchronization measures proposed here have the potential to reduce the age of detection due to the fact that mother-infant communication starts as soon as a baby is born. Importantly, the pattern of synchronization within single brain and/or across brains during face-to-face communication could reveal the risk for ASD prior to the emergence of behavioral symptoms.

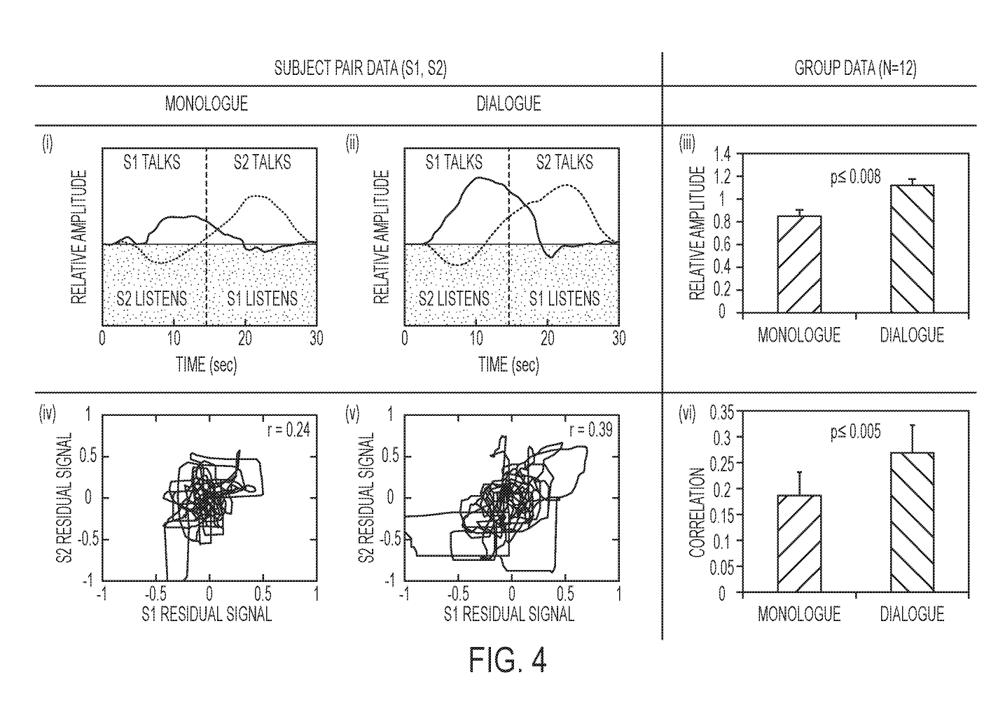

The face-to-face language paradigm proposed herein represents a shift of paradigm to investigate the emergence of communication in autism in the context of interpersonal interactions. It has been proposed that development of communication requires social interactions across individual brains, and that the interaction between them enables specific mechanisms for brain-to-brain coupling. fMRI investigations recording signals from a speaker, and later recording signals from a listener showed that both brains exhibit joint temporally coupled response patterns. Using hyperscanning (simultaneous scanning of two subjects), Jiang and colleagues observed increased synchronization in the left inferior frontal gyms during face-to-face dialogue. Jiang et al., “Neural synchronization during face-to-face communication,” 32(45) J. Neuroscience 16064-69 (2012). Likewise, gestural communication also induces brain-to-brain coupling. Applicant's preliminary studies of cross-brain interactions during face-to-face communication between two healthy adults that performed a normal conversation (dialogue condition, red line) and alternated listening and talking (nondialogue condition, blue line) are depicted in FIG. 4.

The recordings are from left dorsolateral prefrontal cortex (BA 46) for both subjects (Subject 1 and Subject 2) as determined by normalization of digitized probe position to the Montreal Neurological Institute (MNI) standard brain space using NIRS-SPM software. The fNIRS signals from both individuals (left panel) are anticorrelated as observed by the crossing of the subjects' signals corresponding to the opposite roles of the subject at a given time (listening versus talking). The coherence between two people in these areas during communication was −0.83 during dialogue and −0.34 during monologue conditions. When the task components are removed and the coherence is calculated from/with the residual signal only (bottom row), the coherence between signals corresponding in time for the two subjects remains higher for the dialogue condition (r=0.39) than for the monologue condition (r=0.24).

Applicant proposes a novel hypothesis related to the synchronization of mother and infant brain systems during interaction to predict social and communication disabilities in ASD, and to inform an early and objective clinical biomarker for autism. During the first year of life, babies' pre-linguistic skills are shaped by face-to-face interaction and turn taking with their caregivers. Furthermore, early behavioral interventions seem to ameliorate early language and communication symptoms of ASD suggesting that social interactions could have an impact on the development of the neural circuits involved. Although ASD is characterized as a disorder with severe communication deficits, previous investigations on the neural substrate for communication have focused on the autistic subject only and the role of interpersonal interaction for communication development has not been studied.

Aspects of the invention use fNIRS to simultaneously record neural activity during child/parent interactions consisting of blocks of communication (speech and song) interspersed with rest periods of no communication. The paradigm will allow not only the study of brain activity on awake behaving infants but also their synchronizations with a parent or familiar caregiver. Embodiments of the invention are expected to provide a platform for future investigations of the neural mechanisms of interpersonal interaction in autism and represent a departure from current diagnostic criteria for ASD by incorporating the mother and baby behavioral interaction with their cross-brain synchronization in real-time. This innovative approach to ASD diagnosis could reduce the dependence on purely behavioral and subjective diagnostic markers, as well as advance models of the neural biology that underlies the development of interpersonal interactions.

### System Overview

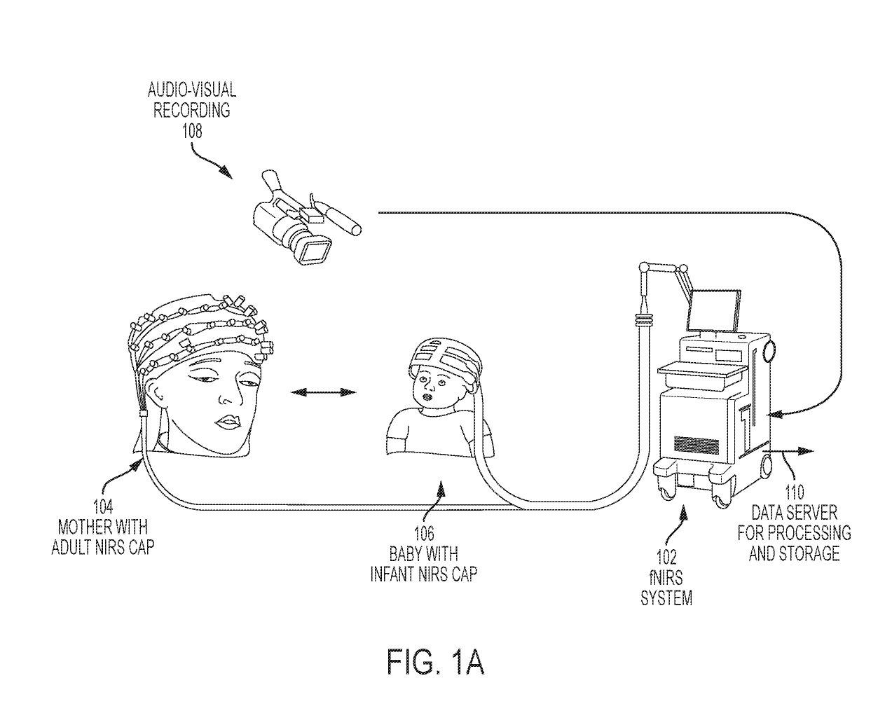

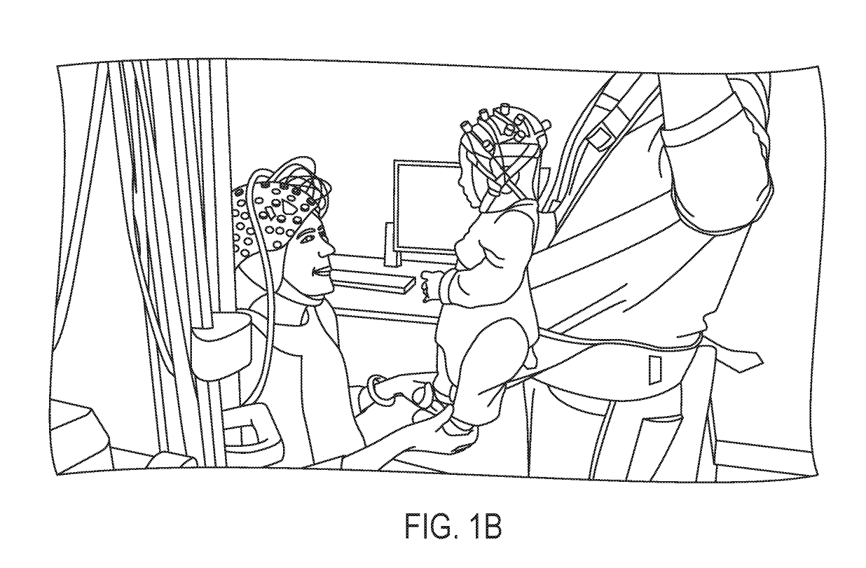

Referring now to FIG. 1A, one aspect of the invention provides a system 100 including a functional near-infrared spectroscopy (fNIRS) system 102, a first near-infrared spectroscopy (NIRS) cap 104 (e.g., sized for an adult), a second NIRS cap 106 (e.g., sized for an infant, toddler, or young child), an audio-visual recording device 108, and a computing device 110 programmed to receive, store, and/or process data generated by any of the other components 102, 104, 106, 108 in the system 100.

Portions of system 100 (namely, first NIRS cap 104 and second NIRS cap 106) are depicted in use by a mother and infant child in FIG. 1B.

fNIRS is an optical technique for monitoring tissue oxygen saturation, changes in hemoglobin volume and, indirectly, brain blood flow and muscle oxygen consumption. There are several benefits of employing fNIRS over other more traditional brain recording techniques such as fMRI. First, fNIRS allows subjects to behave in a more natural way while undergoing a scan and allows for recording of concurrent behavioral and cortical activities. Next, fNIRS can employ spatially accurate, multiple channel recordings of the cortex, which can be observed and manipulated through the behavior of subjects in real-time. Furthermore, the fNIRS methodology is appropriate for infants and young children, elderly, and patients with psychoneurological problems because it is non-invasive and requires little constraining of subject's movement, thus eliminating the need for sedation typical of fMRI. Importantly, fNIRS is more available readily and relatively inexpensive compared to other non-invasive medical devices like MRI or more invasive medical devices like PET, and thus, has the potential to improve access to early intervention. In addition, the temporal resolution of fNIRS is significantly higher than that of fMRI. Correspondence between fNIRS and simultaneously acquired fMRI signals is approximately r≈0.8. Finally, fNIRS allows for simultaneous recording of two awake behaving subjects making it an optimal technique for interpersonal interaction studies.

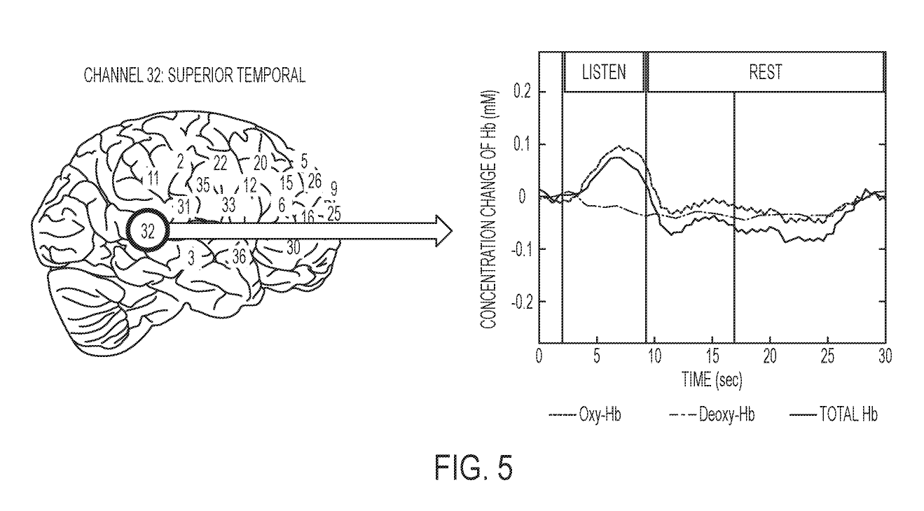

Previous fMRI studies by Applicant have shown strong positive BOLD signal in language regions using alternating epochs of silence and speech. Because fNIRS also measures the BOLD signal, embodiments of the invention use paradigms with the same basic principle of alternating epochs of speech/song (activation) and epochs of silence (rest) to calibrate the system to detect Wernicke's and Broca's Areas activations. For example, three near infrared signals can be simultaneously measured corresponding to oxyhemoglobin, deoxyhemoglobin and total hemoglobin concentrations as discussed herein. Preliminary experiments using a 52-channel (optodes) Hitachi ETG-4000 system located in the Child Study Center at Yale University School of Medicine show recordation of expected near-infrared signals with this paradigm. FIG. 5 depicts the position of the 52 channels (yellow dots) in a standardized brain volume. Using multiple 15-second epochs of silence and 15 seconds of passive listening to sentences, aspects of the invention are able to detect an increase of hemoglobin concentration in the superior temporal cortex (channel 32) during speech compared to rest in a representative healthy adult. Similar findings are also observed on the NIRSOPTIX™ CW6™ system NIRS system in the Haskins Laboratory at Yale University.

fNIRS systems 102 and NIRS caps 104 and 106 are commercially available from a variety of sources including Rogue Research Inc. of Montreal, Quebec; NIRx Medical Technologies, LLC of Los Angeles, Calif.; TechEn, Inc. of Milford, Mass.; Cortech Solutions, Inc. of Wilmington, N.C.; Shimadzu Corporation of Kyoto, Japan; and Hitachi Medical Systems America Inc. of Twinsburg, Ohio.

Components 102, 104, 106, and 108 can operate in their normal manner with further processing in accordance with embodiments of the invention occurring on the computing device 110.

Method of Identifying an Individual Afflicted with a Social Disorder

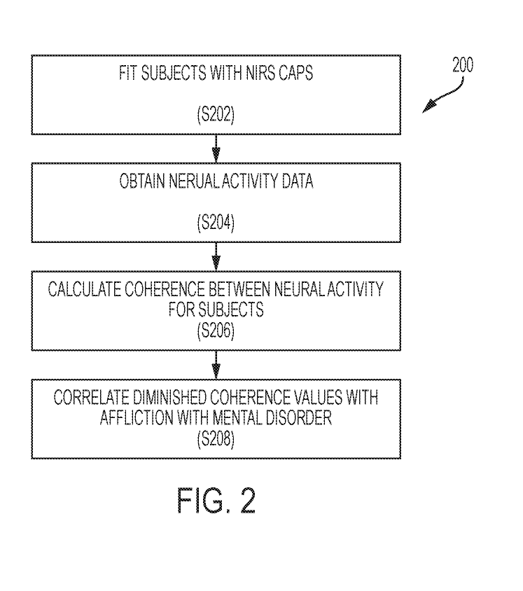

Referring now to FIG. 2, another aspect of the invention provides a method 200 of identifying an individual afflicted with a social disorder.

In step S202, two subjects are fitted with NIRS caps (e.g., in accordance with manufacturer instructions). For example, the fNIRS surface optodes can be positioned in similar head locations on the mother and baby to obtain cortical signals of corresponding brain regions. The neural activity data can include fNIRS signals collected using the system 100 described herein. For example, changes in oxygenated hemoglobin (oxy-Hb) and deoxygenated hemoglobin (deoxy-Hb) concentrations can be measured using the same apparatus and methodology described in H. Bortfeld et al., “Identifying cortical lateralization of speech processing in infants using near-infrared spectroscopy,” 34(1) Dev. Neuropsychol. 52-65 (2009). A suitable fNIRS system includes the NIRSOPTIX™ CW6™ system available from TechEn, Inc. of Milford, Mass.

Laser diodes measure the changes in oxygenated hemoglobin (oxy-Hb) and deoxygenated hemoglobin (deoxy-Hb) concentrations. For each channel, the absorption of near-infrared light at 780, 805, and 830 nm wavelengths can be measured with a 10 Hz sampling frequency. These wavelengths are used by SHIMADZU® devices. Other NIRS devices can use other wavelengths and obtain the same or similar results. For example, Applicant understands that HITACHI® devices utilize only two wavelength, which are calculated to maximize differences in molecular absorption coefficients for oxy-Hb and deoxy-Hb on each side of the point around 805 nm at which oxy-Hb and deoxy-Hb have similar molecular absorption coefficients.

Each channel consists of a pair of emitter and detector probes. The alternating emitters and detectors pairs, at a distance of 3 cm from each other for adults and 2.0 cm for infants are set in a cap that is flexible to adjust to the head surface to maximize skin contact and support more stable measurements.

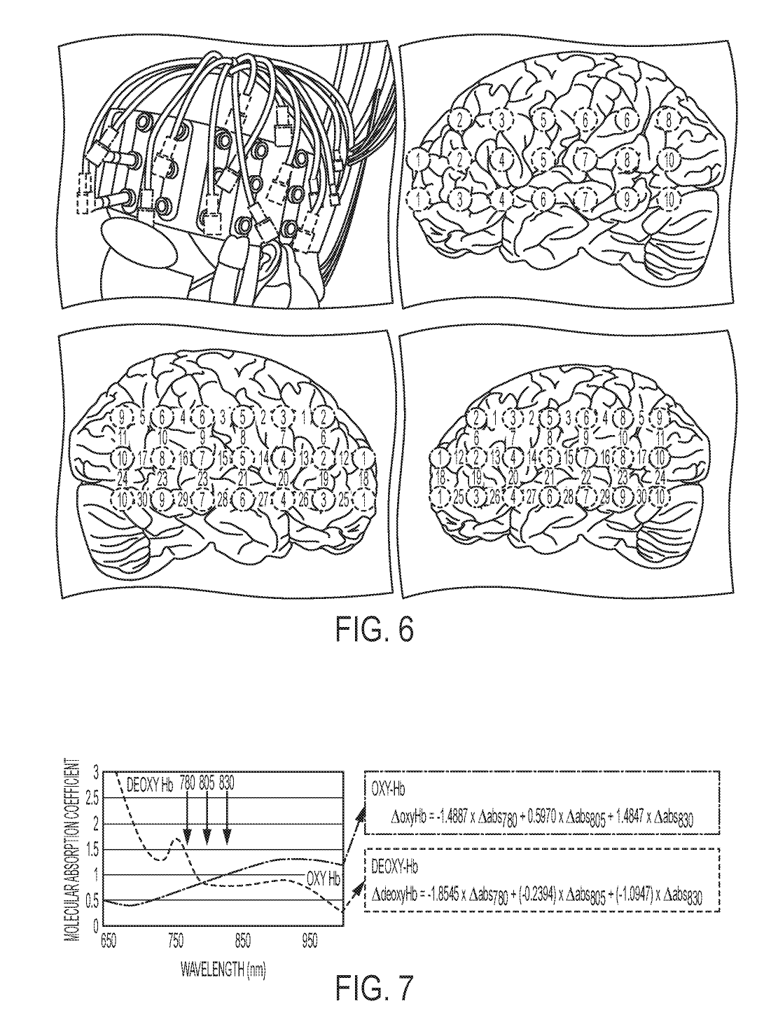

The probes can be positioned on each participant's head, aligned to the midline defined as the arc running from the nasion through Cz to the inion. The position of the probes can be based on the 10-20 international coordinate system described in H. H. Jasper, “Report of the committee on methods of clinical examination in electroencephalography: 1957,” 10(2) Electroencephalography & Clinical Neurophysiology 370-75 (1958) and International Publication No. WO 2014/152806, which provides an accurate relationship with the cortical anatomy using the onsets of the zygomatic bones as preauricular points. The lowest and most anterior optode can be placed at Fpz. The lowest optode row can be aligned with the line connecting Fpz-T3 (frontal to temporal). The channels can be placed over the forehead, the temporal lobes, the parietal and the visual regions as depicted in FIG. 6.

A 3D magnetic digitizer (available under the PATRIOT™ from Polhemus of Colchester, Vt.) can be used to identify the optode position of each subject immediately before data collection to normalize the position of the individual channels of the NIRS cap to the shape of each subject's skull as discussed in M. Okamoto & I. Dan, “Automated cortical projection of head-surface locations for transcranial functional brain mapping,” 26 Neuroimage 18-28 (2005). Three-dimensional coordinates of anatomical landmarks on the head can be recorded in addition to locations of the individual optodes using procedures previously described in M. Okamoto et al., “Three-dimensional probabilistic anatomical cranio-cerebral correlation via the international 10-20 system oriented for transcranial functional brain mapping,” 21 Neuroimage 99-111 (2004). A digitizer pen can be used to indicate landmark positions of nasion, inion, T3, T4 and Cz according to the standard 10-20 coordinate system. After these anatomical landmarks are recorded, individual probe positions can be obtained. These coordinates can be used to estimate the position of each channel as defined by an emitter-detector optode pair and normalized to Montreal Neurological Institute, MNI, standard brain space coordinates using NIRS-SPM, a MATLAB®-based software program available at http://www.fil.ion.ucl.ac.uk/spm/. The MNI coordinates can be used to calculate probability of channel position using defined Brodmann's Areas and anatomical areas as indicated in the Talairach daemon. The position of the optodes can be optimized to cover auditory cortex and association areas in the left temporal area corresponding to Wernicke's Area associated with speech comprehension, left inferior frontal gyms corresponding to Broca's Area, the prefrontal cortex associated with cognitive control, the parietal cortex associated with the social brain, and the primary visual cortex.

In step S204, neural activity data is obtained for a first subject and a second subject during verbal interaction between the first subject and the second subject. The data can be obtained through various methods and channels.

For example, the computing device 110 can include the appropriate hardware and/or software to implement one or more of the following communication protocols: Universal Serial Bus (USB), USB 2.0, IEEE 1394, Peripheral Component Interconnect (PCI), Ethernet, Gigabit Ethernet, and the like. The USB and USB 2.0 standards are described in publications such as Andrew S. Tanenbaum, Structured Computer Organization Section § 3.6.4 (5th ed. 2006); and Andrew S. Tanenbaum, Modern Operating Systems 32 (2d ed. 2001). The IEEE 1394 standard is described in Andrew S. Tanenbaum, Modern Operating Systems 32 (2d ed. 2001). The PCI standard is described in Andrew S. Tanenbaum, Modern Operating Systems 31 (2d ed. 2001); Andrew S. Tanenbaum, Structured Computer Organization 91, 183-89 (4th ed. 1999). The Ethernet and Gigabit Ethernet standards are discussed in Andrew S. Tanenbaum, Computer Networks 17, 65-68, 271-92 (4th ed. 2003).

In other embodiments, computing device 110 can include appropriate hardware and/or software to implement one or more of the following communication protocols: BLUETOOTH®, IEEE 802.11, IEEE 802.15.4, and the like. The BLUETOOTH® standard is discussed in Andrew S. Tanenbaum, Computer Networks 21, 310-17 (4th ed. 2003). The IEEE 802.11 standard is discussed in Andrew S. Tanenbaum, Computer Networks 292-302 (4th ed. 2003). The IEEE 802.15.4 standard is described in Yu-Kai Huang & Ai-Chan Pang, “A Comprehensive Study of Low-Power Operation in IEEE 802.15.4” in MSWiM'07 405-08 (2007).

The neural activity data can be obtained asynchronously from its collection. For example, the data can be gathered at first time and location, stored, then analyzed at a second time and location that can be different than the first time and location.

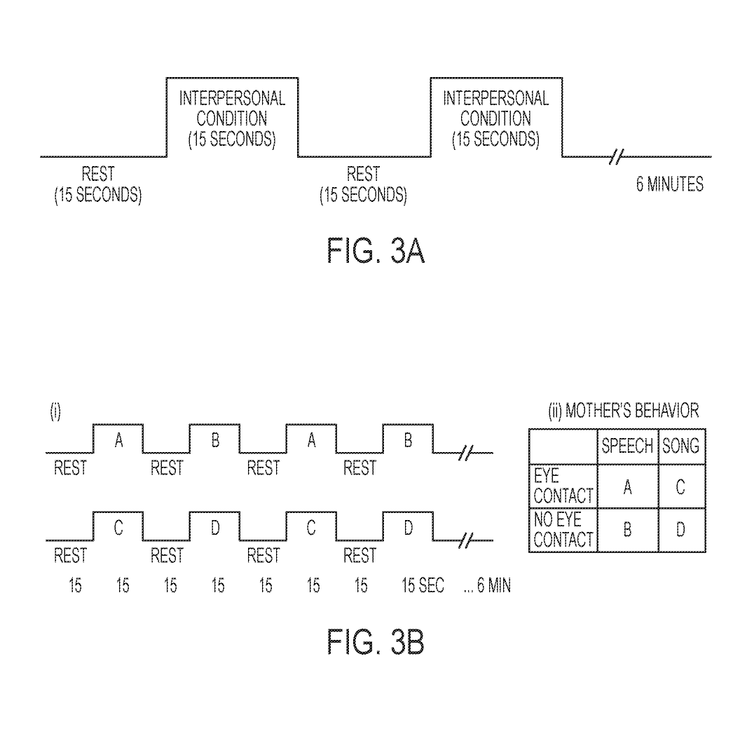

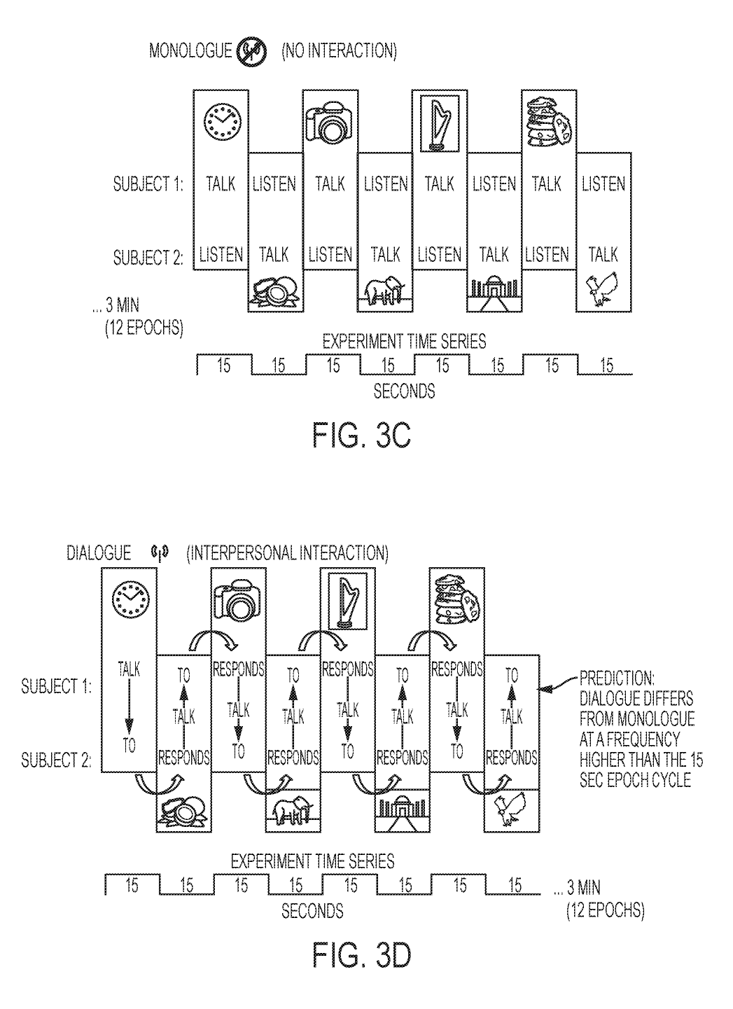

The verbal interaction can include speaking and/or singing. In some embodiments, the verbal interaction includes alternating epochs of verbal tasks and rest as depicted in FIGS. 3A-3D. For example and as depicted in FIG. 3A, an initial rest session (e.g., about 6 minutes) can be followed by a session (e.g., about 6 minutes) of alternating epochs (e.g., about 15 seconds) of rest and interpersonal communication.

In another embodiment, the subject acting as a “speaker” in a pair of adults can be instructed to talk to the “listener” about events in the previous day. The listener can be instructed to attend to the narrative without answering or making non-verbal sounds, and will be later asked questions to ensure task compliance.

One subject (the speaker) can be instructed to speak to the other subject (the listener) using simple sentences and/or avoiding non-verbal sounds. The listener (e.g., an infant) may babble, talk, smile, and the like without impairing the test.

In still another embodiment depicted in FIG. 3B, sessions can start with an initial 6 minutes scan of free mother-child interaction that will allow the participants to became adjusted to the apparatus, followed by other 6 minutes of baseline state with no interaction between mother and child. The speech and song conditions, with and without mother's eye contact, alternating with 15 seconds of silence (rest) can follow. During the mother-infant interaction, only the mother's behavior can be controlled; the baby's vocalization or verbalizations and gaze will be recorded as well as their timing with respect to the mother's speech to aid in the interpretation of the brain data. The mother/parent will be instructed to speak to baby in a familiar manner including the use of “mothereze” if typical for the subject pair.

In step S206, coherence between the neural activity data for the first subject and the neural activity data for the second subject can be calculated.

In one embodiment, signals reflecting changes in oxy-Hb, deoxy-Hb, and total-Hb concentrations are calculated using a modified Beer-Lambert approach as described in M. Cope et al., “Methods of quantitating cerebral near infrared spectroscopy data,” 222 Adv. Exp. Med. Biol. 183-89 (1988) and as depicted in FIG. 7. To avoid NIRS path-length issues, oxy-Hb data from each channel can be normalized by linear transformation so that mean±SD of oxy-Hb levels during the initial 5 seconds of rest will be “zeroed”. Data of hemodynamic signals from individual channels can be low-pass-filtered through a 25-point Savitzky-Golay filter as described in A. Savitzky and M. J. E. Golay, “Smoothing and Differentiation of Data by Simplified Least Squares Procedures,” 36(8) Analytical Chemistry 1627-39 (1964), baseline corrected, and detrended to remove system drift. Channel artifacts found upon visual inspection can be corrected by linear interpolation of the first and last data points around the artifact or by ICA. The molecular extinction coefficient of oxy-Hb, deoxy-Hb, and total hemoglobin detected by fNIRS can be calculated using Platform for Optical Topography Analysis Tools (POTATo) available from the Research & Development Group of Hitachi, Ltd. of Tokyo, Japan at http://www.hitachi.co.jp/products/ot/analyze/kaiseki_en.html for use within MATLAB® 7.0 software available from The MathWorks, Inc. of Natick, Mass. The POTATo™ software also converts each channel position into Montreal Neurological Institute (MNI) normalized brain space as described in Okamoto and Dan, 2005. To improve the sensitivity of the fNIRS signal, physiological noise components can be removed using independent component (ICA) and global systemic peripheral physiological measurements of respiration and HR as described in E. Kirilina, “The physiological origin of task-evoked systemic artifacts in functional near infrared spectroscopy,” 61 Neuroimage 70-81 (2012).

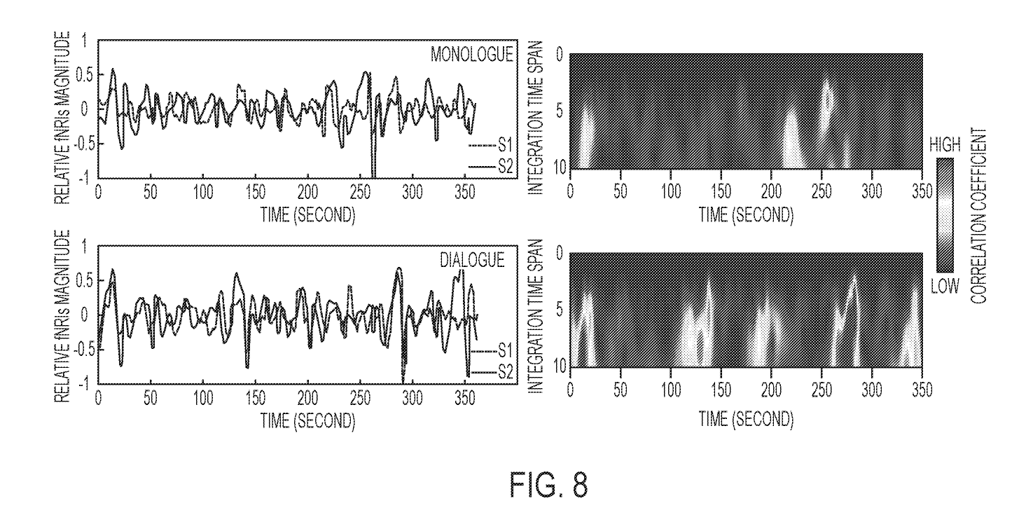

The synchronization between fNIRS signals and each pair of subjects can be analyzed by cross-correlation analysis and wavelet coherence analysis using the residual signal by removing task driven effect from the raw data. The task driven effect can be removed from the NIRS signal by subtracting the expected BOLD signal from the raw data. The expected BOLD signal model can be derived by convolving the 15-second on and 15-second off time series and the hemodynamic response function provided by the Statistical Parametric Mapping (SPM08) software available from the Wellcome Trust Centre for Neuroimaging at http://www.fil.ion.ucl.ac.uk/spm/. First, a cross-correlation method can be employed taking into account the possibility of a variable delay for the global coherence measure. Wavelet transform coherence (WTC) analysis described in C. Torrence & G. P. Compo, “A Practical Guide to Wavelet Analysis,” 79 Bulletin of the American Meteorological Society 61-78 (1998) can also be performed on the residual signal for instantaneous coherence measure as discussed in X. Cui et al., “NIRS-based hyperscanning reveals increased interpersonal coherence in superior frontal cortex during cooperation.” 59(3) Neuroimage 2430-37 (2012) and A. Grinsted et al., “Application of the cross wavelet transform and wavelet coherence to geophysical time series,” 11(5/6) Nonlinear processes in geophysics (2004). The data previously shown in FIG. 4 can be analyzed with WCA as shown in FIG. 8.

The residual signals for the dorsolateral frontal cortex for subjects 1 and 2 are shown in the left panels of FIG. 8 for monologue and dialogue conditions. The coherence between the residual signals for each condition (right panels) is represented along the time course of the experiment (x-axis) according to the color scale. Hot colors represent high correlation and cool colors represent low correlations. The y-axis indicates the integration time span (related to the frequency of the components) from 0 to 10 second. Note that the communicating subjects demonstrate higher episodic synchrony (bright colored vertical patches) during the dialogue condition than during the monologue condition. Convergence of the results between both analyses will be evidence for brain-to-brain synchrony during interpersonal interaction.

In step S208, diminished coherence values are correlated with affliction with the social disorder.

### Results and Feasibility

### Coherence Analysis of Cross-Brain Synchronization

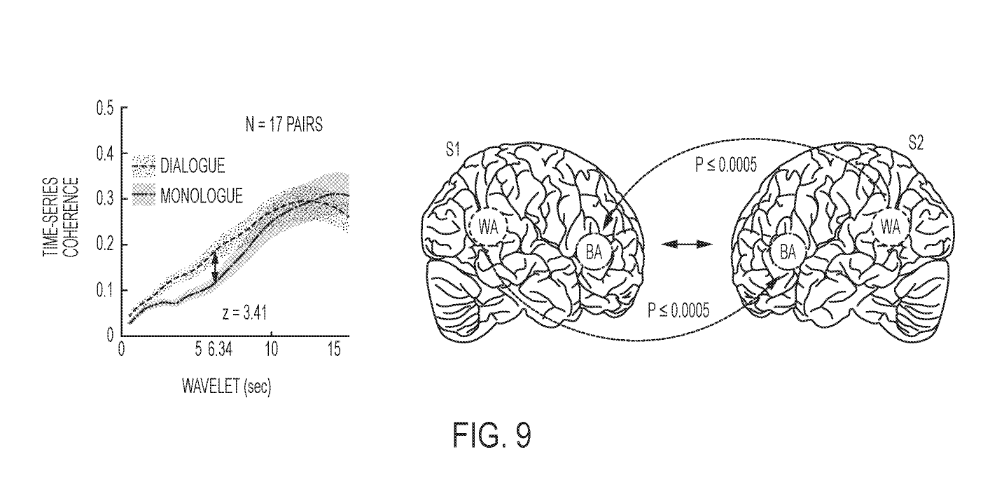

FIG. 9 depicts coherence data acquired using a 52-channel SHIMADZU® NIRS system. Coherence is plotted for the deoxy-Hb fNIRS signals of Wernicke's and Broca's Areas during monologue (blue line) and dialogue (red line) conditions for the group of 17 subject pairs. This result indicates a significantly higher synchrony during the dialog condition than the monologue condition at a frequency of 6.4 seconds (p<0.005). Note that the communicating pairs of subjects demonstrate highest synchrony during the change of epochs (15 seconds) that does not differ between the dialogue and monologue conditions. However, the difference is observed nearly midway between the epochs (at wavelength of 6.34 secs) as predicted in FIG. 3D. This observation, however, was observed only for the face-to-face condition where subject pairs were facing each other during the interactions and was not observed in the occluded condition. Findings are bilaterally significant across pairs of subjects and unbiased with respect to regions of interest. These findings of coherence are specific to Broca's and Wernicke's areas across the two participating brains. fNIRs signal from Wernicke's area (WA, blue circles) coheres with signals from Broca's area (BA, pink circles) of the partner subject during face to face interaction.

### Adult Pilot Studies That Document The Role Of Faces In Coherence Measures

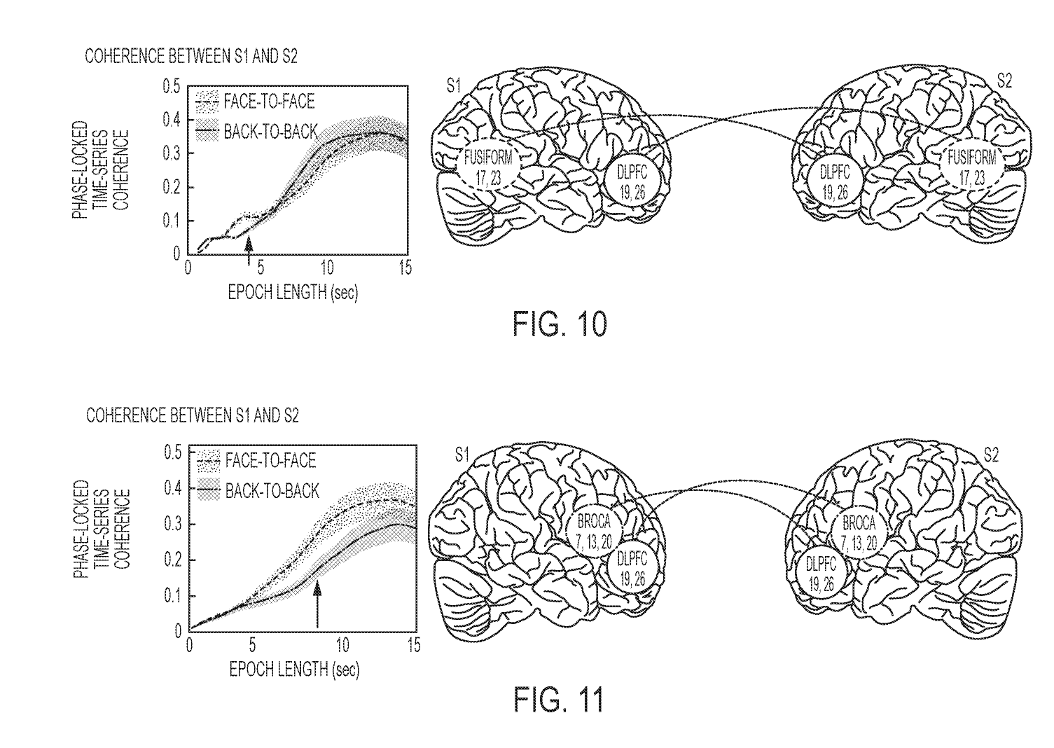

Referring now to FIG. 10, pilot studies compared face-to-face versus occluded conditions during monologue and dialogue tasks (n=17 pairs), and revealed significant differences in coherence between the pairs of subjects in the face-to-face condition at a wavelet of 4.22 seconds, p≦0.005. In particular, this difference was observed between the fusiform gyms and the dorsolateral prefrontal cortex. As in the case of the previous findings for the monologue versus dialogue experiment, this significant coherence was observed bilaterally between both brains. As the fusiform gyms is well-known for its functional role in face processing, this finding was not surprising. However, the coherence with the dorsolateral prefrontal cortex suggests that face information may be incorporated into control mechanisms associated with interpersonal communication.

The second finding in the face-to-face versus occluded condition is illustrated in FIG. 11 showing a coherence difference between pairs of subjects at a wavelet (Epoch length) of 8.42 seconds between the dorsolateral prefrontal cortex and Broca's Area, p≦0.001. The coherence with dorsolateral prefrontal cortex and the well-known speech production area, Broca's Area, suggests a functional connection between control and regulatory systems and speech production systems. These pilot findings suggest a significant role for mechanisms specialized for face processing (reception), speech production (transmission) and control mechanisms (regulation) during interpersonal communication.

Dynamic Incorporation of Face Information into Transmission and Receptive Language Processes During Interpersonal Communication

Social interaction and direct communication between two or more individuals are fundamental human functions that can be investigated with simultaneous acquisitions of BOLD signals from two interacting subjects using functional near-infrared spectroscopy (fNIRS). Applicant tested the hypothesis that eye-to-eye information is dynamically incorporated into this receptive/transmission system during interactive communication. If so, then findings would be supportive of a mechanism whereby eye-to-eye contact and spoken information are integrated within the canonical language system.

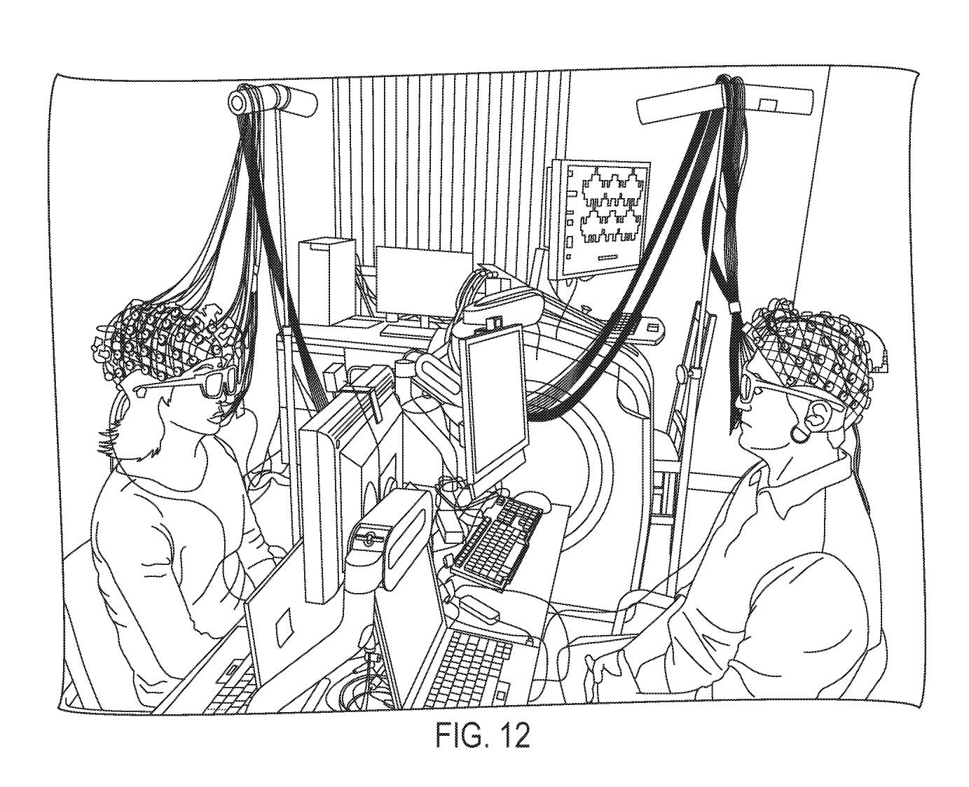

BOLD signals were acquired using a whole head fNIRS system consisting of 84 channels divided evenly between two interacting subjects (i.e., 42 channels per subject). Signals were acquired at a temporal resolution of 27 milliseconds with a spatial resolution of 3 cm using a SHIMADZU® LABNIRS® system, and synchronized with simultaneous dual-brain eye tracking glasses (SMI™ ETG 2 Wireless) having 0.5° accuracy as depicted in FIG. 12.

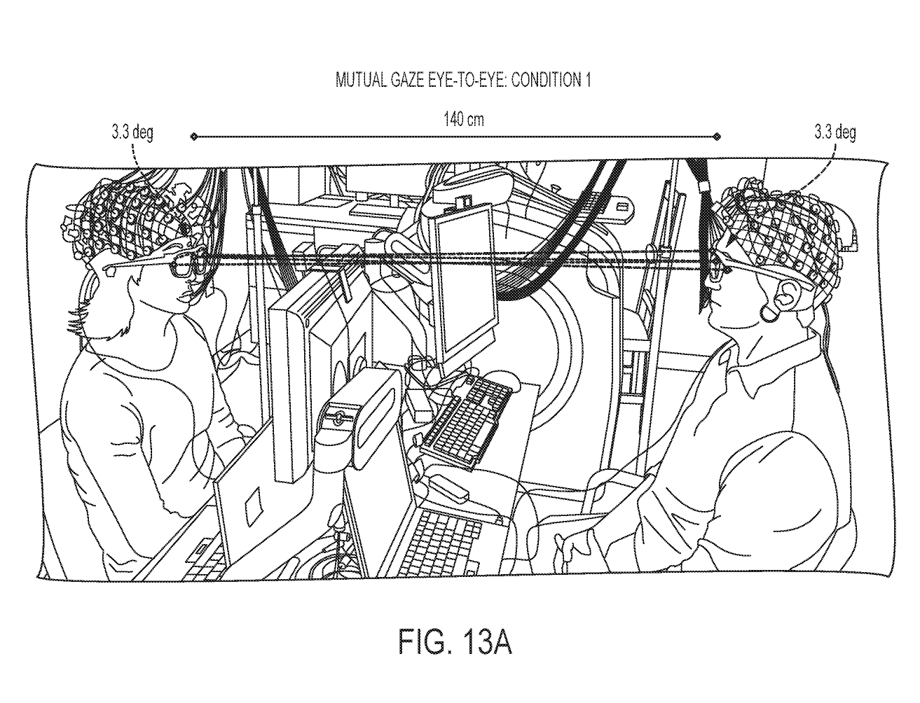

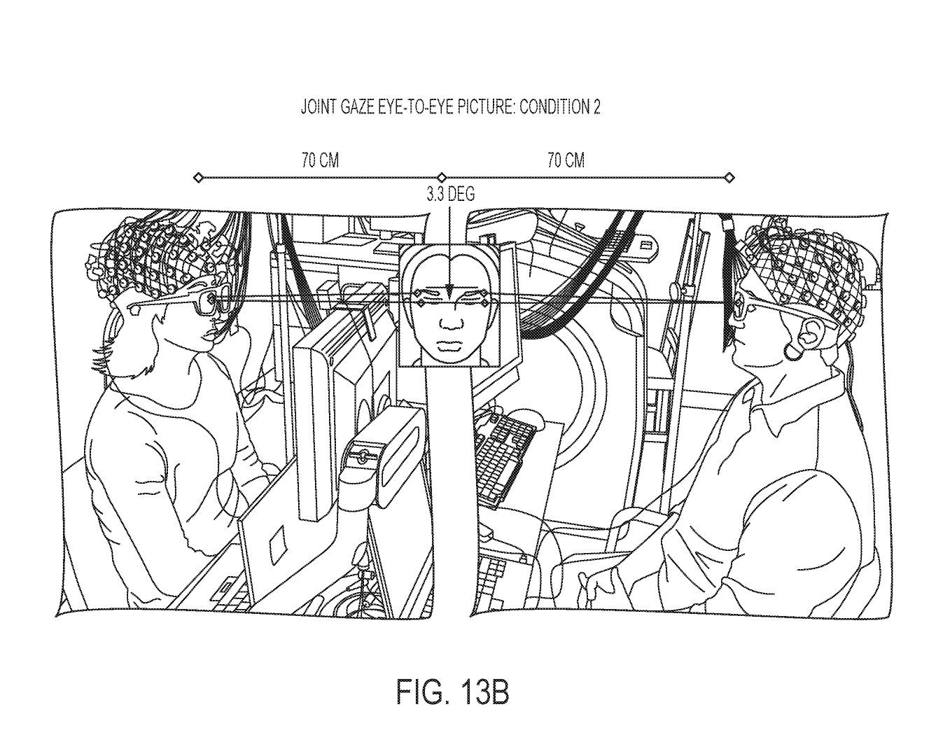

Dyads of 15 subjects participated under two conditions: mutual-gaze eye-to-eye contact (depicted in FIG. 13A) and joint-gaze eye-to-picture contact (depicted in FIG. 13B) using alternating 30 second epochs consisting of an 18 second active period and a 12 second rest period. The active period consisted of 3 cycles on and off the eye target. During the rest period, subjects focused on a crosshair target separated from the faces by 10°.

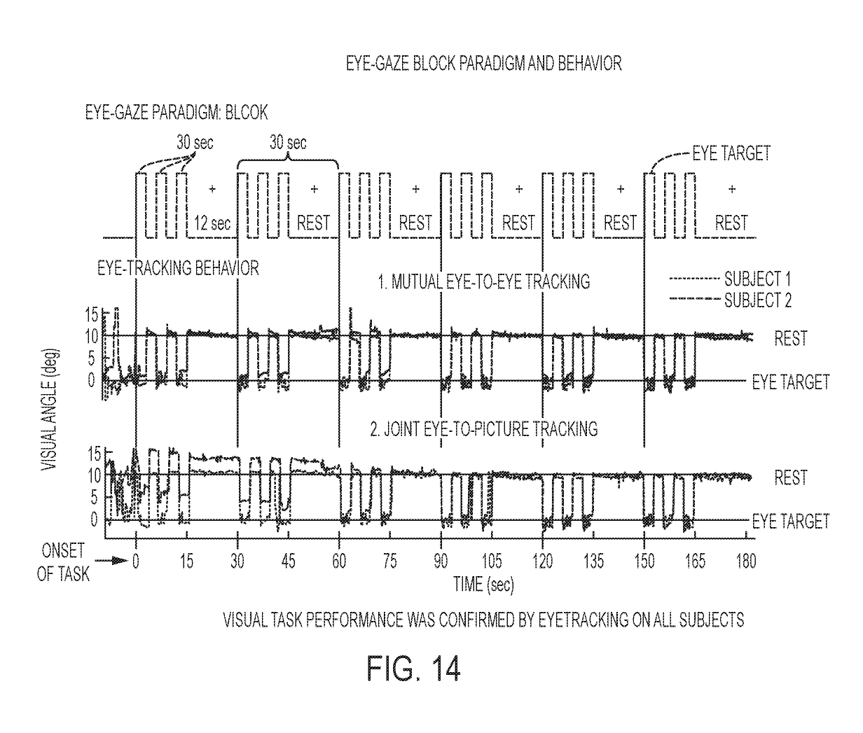

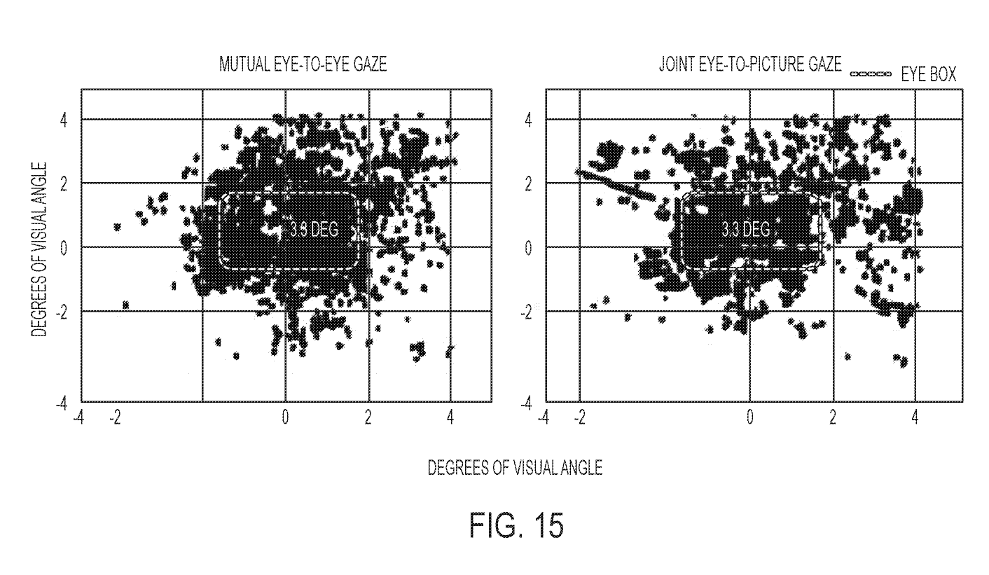

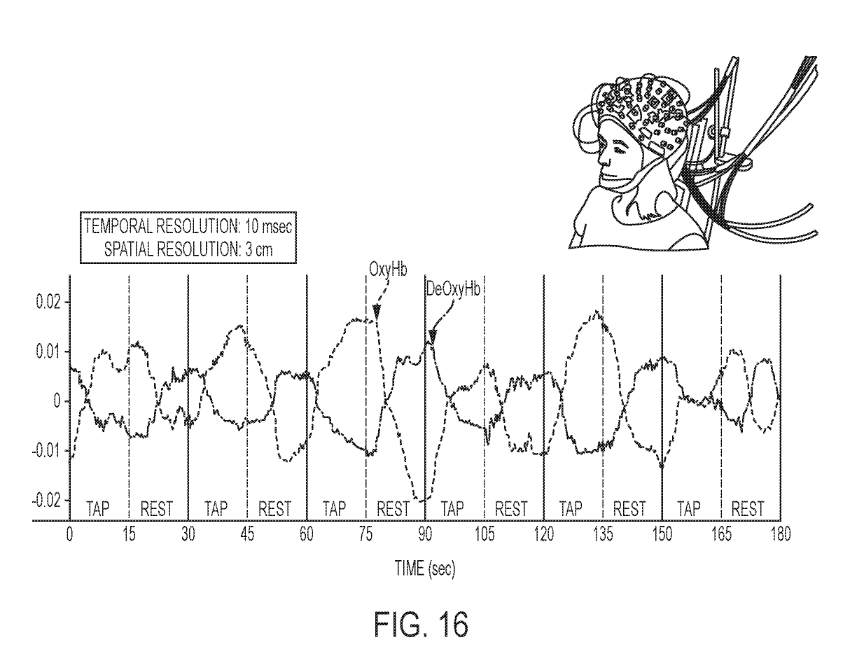

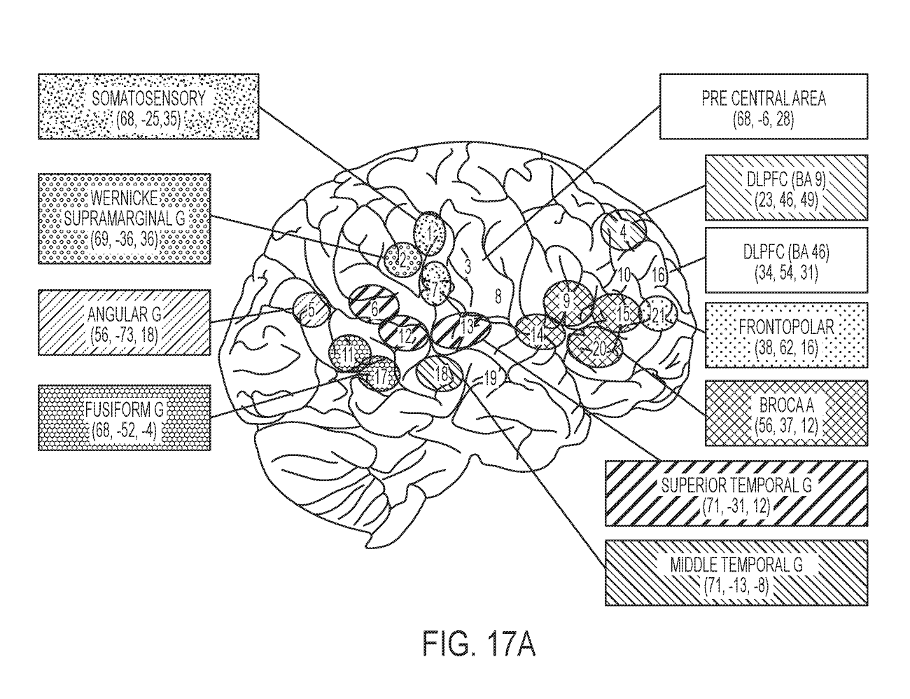

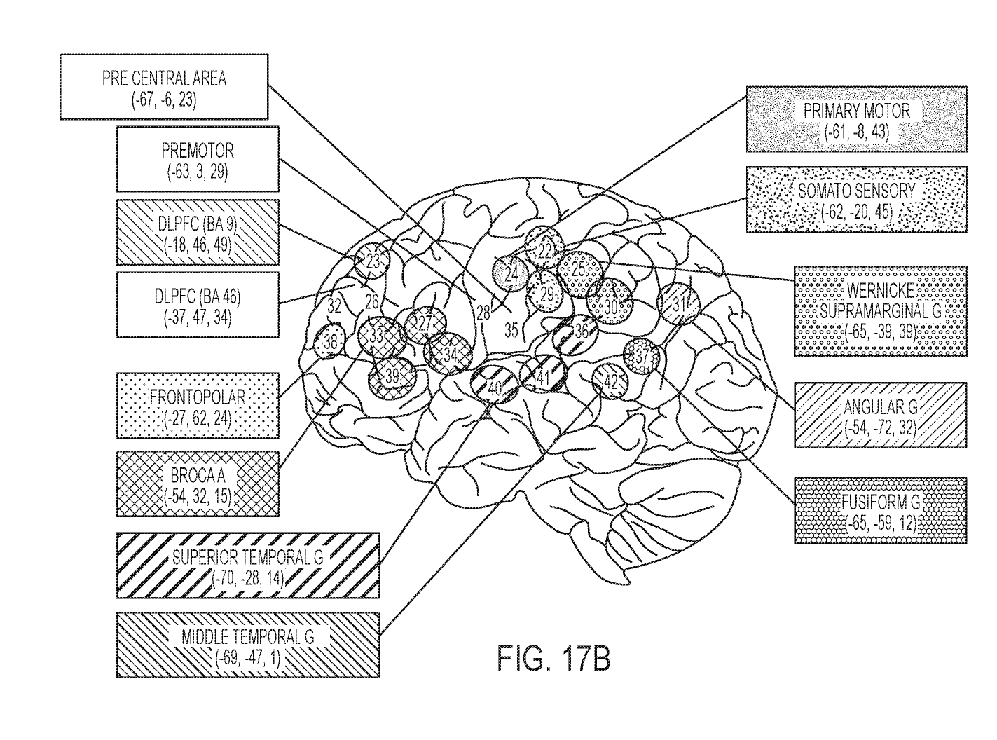

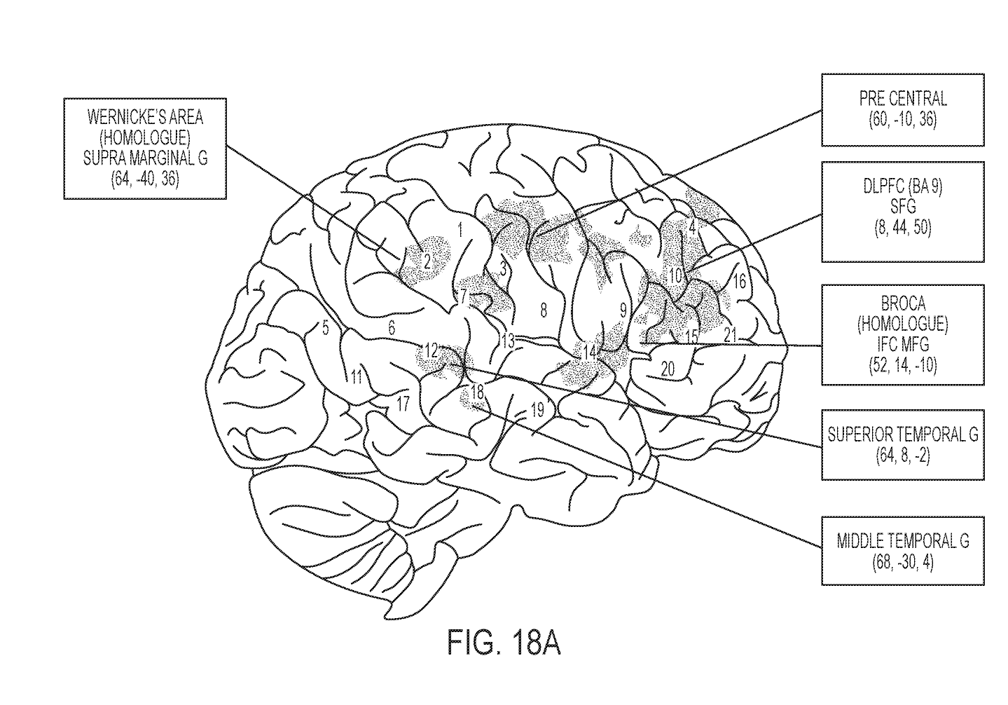

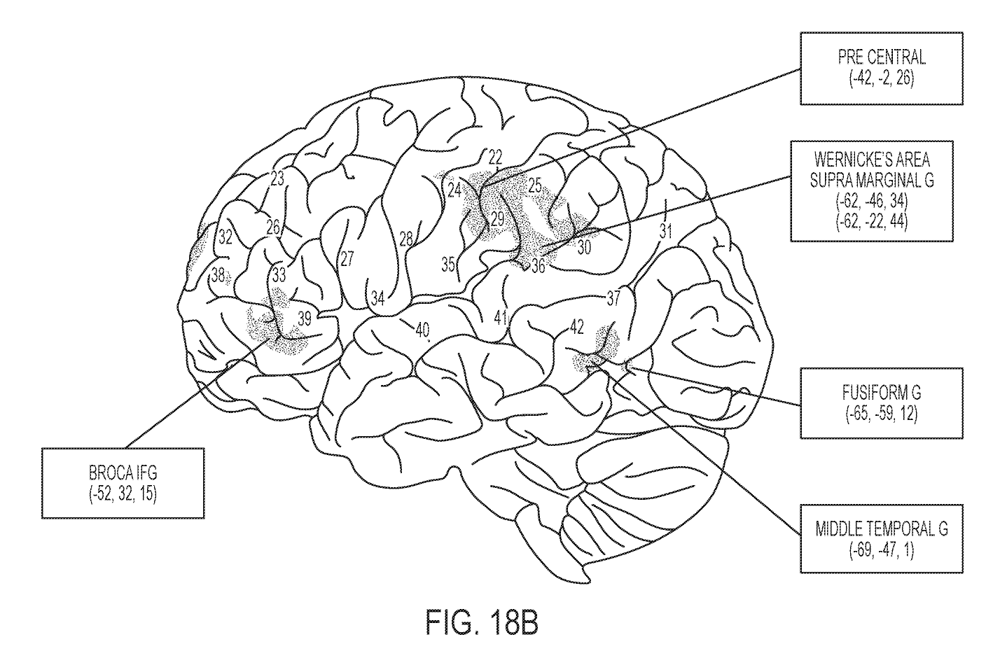

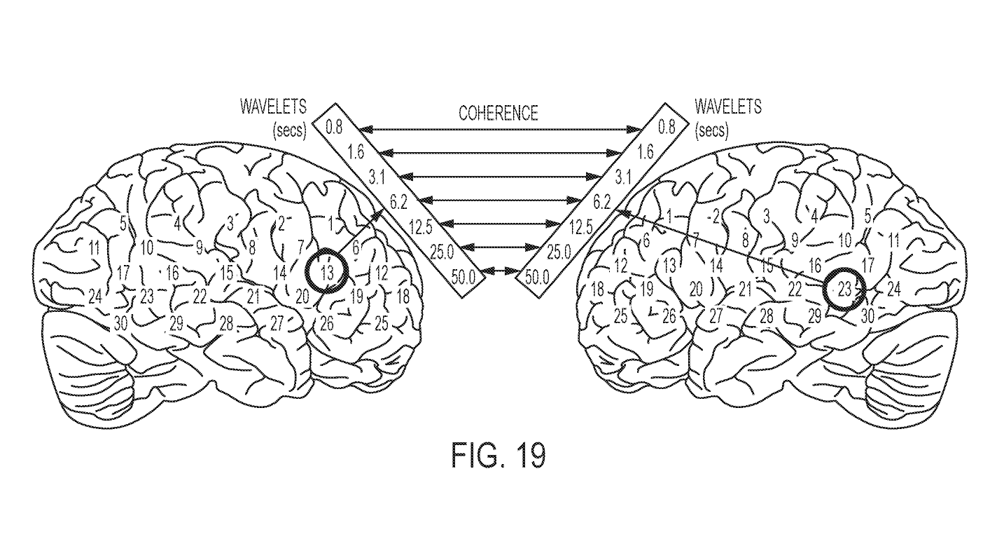

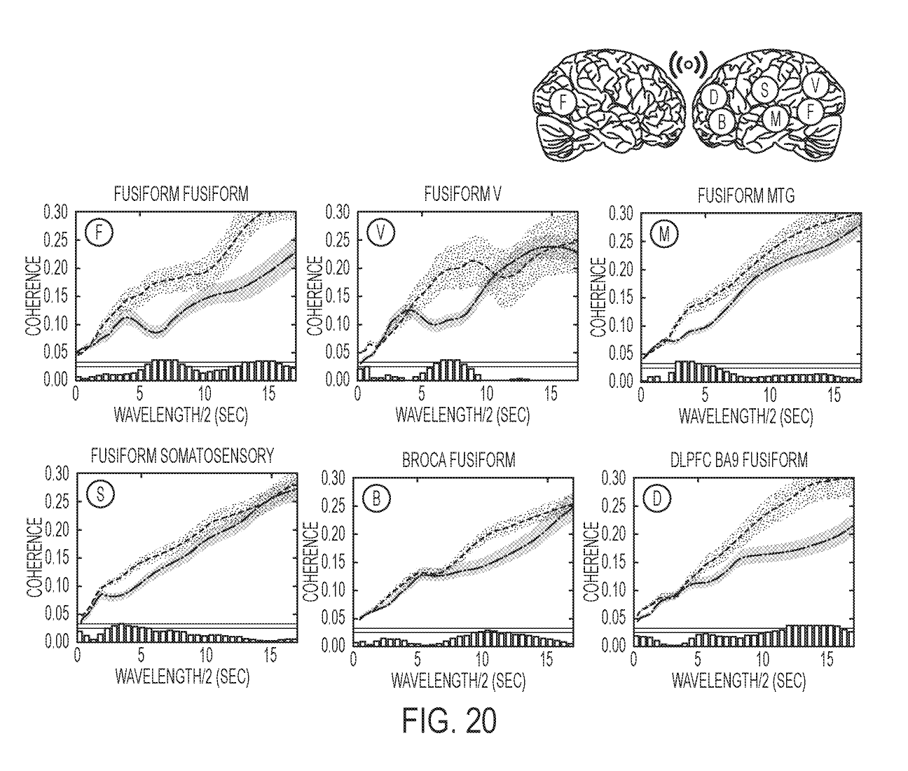

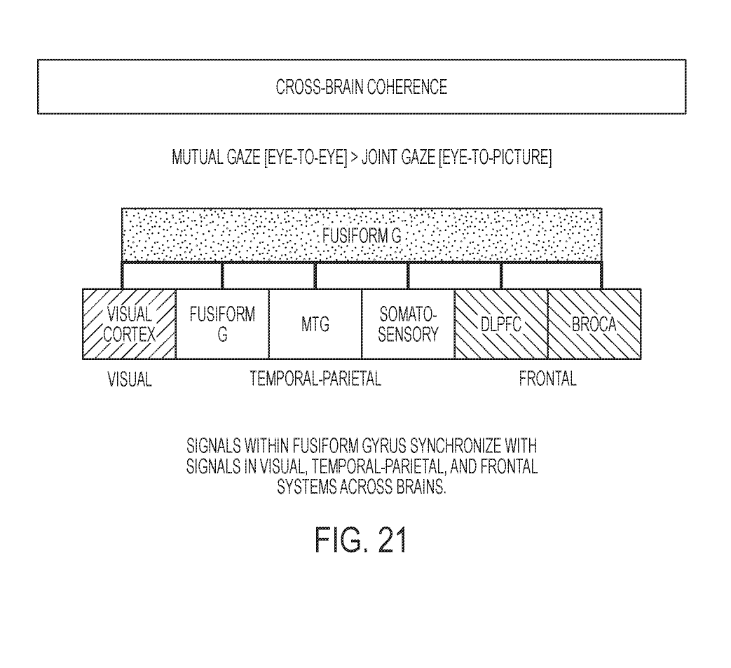

Task performance and compliance were confirmed by eye-tracking with no evidence for performance differences between the two conditions as depicted in FIGS. 14 and 15. GLM contrast comparisons based on the deOxy Hb signal revealed unique neural activity associated with real-eye-to-eye contact that exceeded eye-to picture contact in canonical language sensitive areas of brain (p<0.01) as depicted in FIGS. 18A and 18B. Wavelet analysis as described in Torrence & Compo was used to quantify coherence between two brains for all pairs of cross-brain regions. Coherence during eye-to-eye conditions exceeded coherence during eye-to-picture conditions (p<0.01) for multiple pairs of cross-brain regions that included fusiform G: visual, temporal-parietal, and frontal regions as well as Wernicke and Broca's Areas. These dual-brain fNIRS imaging and simultaneous eye-tracking findings reveal a theoretical underpinning that unifies eye-to-eye contact and language/communication functions in the brain based on common pathways and integrated systems.

### Analysis of Agreement and Disagreement During Dialogue

Applicant investigated the brain activity of both parties during conversation. A rating scale determined individual attitudes on 40 topics. Participant pairs were selected based on 2 topics of agreement and two topics of disagreement. Participants alternated between talking and listening in 15 second intervals.

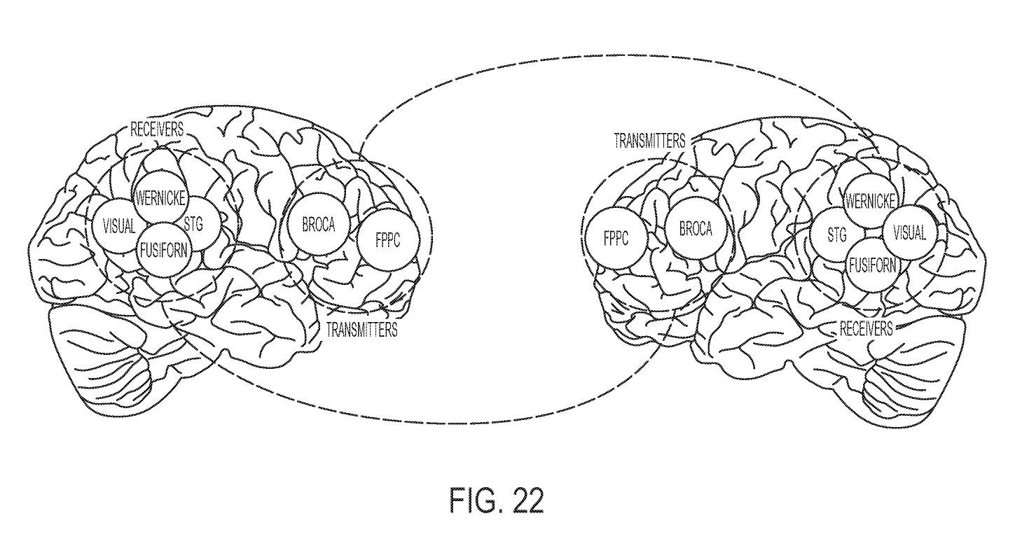

FIG. 22 depicts the cross-brain coherence across brains during dialogue when the subjects are agree. The transmitter and receiver complexes co-vary across brains during dialogue.

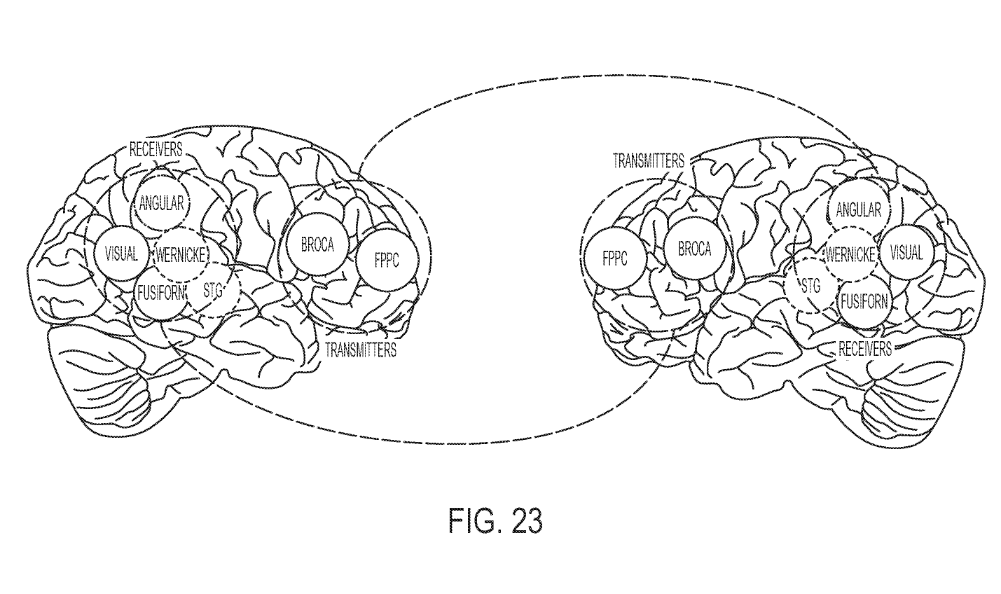

FIG. 23 depicts the cross-brain coherence across brains during dialogue when the subjects are disagree. The transmitter and receiver complexes again co-vary across brains during dialogue. The angular gyms (a social function region) also co-varies. Additionally, the Wernicke and superior temporal gyms (STG) regions of the receiver complex do not co-vary.

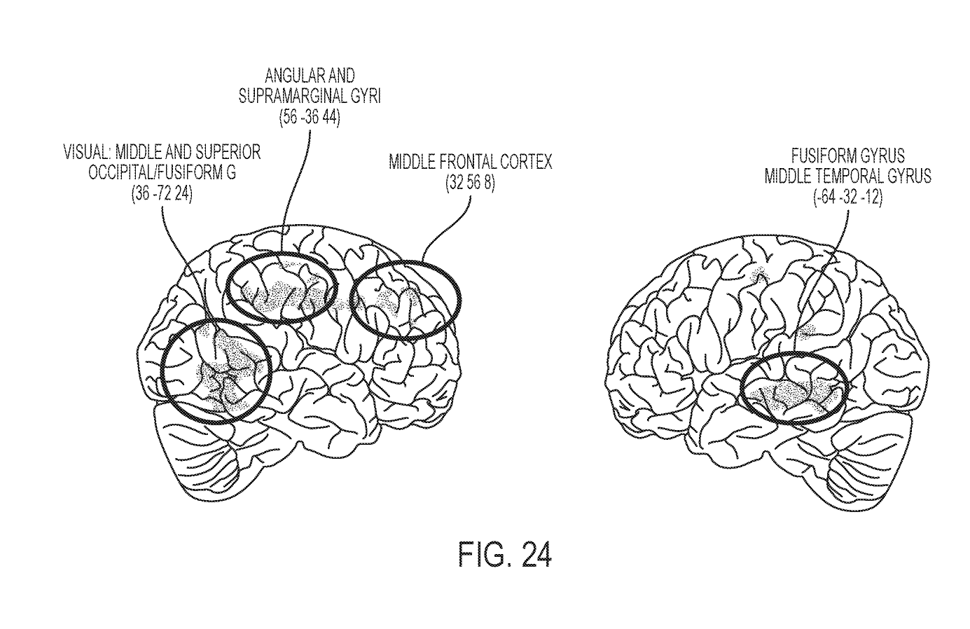

FIG. 24 depicts the single brain results for disagreement relative to agreement. Single-brain findings are consistent with the cross-brain coherence findings. Disagreement involves face processing and the social brain complexes.

### Additional Applications

Although the invention was described in the context of early identification of autism, embodiments of the invention can be applied to identify individuals having other social disorders (e.g., one associated with reduced affinity or social interaction) such as schizophrenia, depression, post-traumatic stress disorder, and the like. Additionally embodiments of the invention can be applied to identify individuals afflicted with a developmental social disorder such as non-specific developmental delay. Additionally, embodiments of the invention can be used to quantitatively assess the effectiveness of a therapy (e.g., early intervention therapies for autism, drugs, and the like) in treating the social disorder. Moreover, embodiments of the invention can be applied to quantitatively assess well-being, facilitate conflict resolution, and the like.

Implementation in Computer-Readable Media and/or Hardware

The methods described herein can be readily implemented in software that can be stored in computer-readable media for execution by a computer processor. For example, the computer-readable media can be volatile memory (e.g., random access memory and the like) and/or non-volatile memory (e.g., read-only memory, hard disks, floppy disks, magnetic tape, optical discs, paper tape, punch cards, and the like).

Additionally or alternatively, the methods described herein can be implemented in computer hardware such as an application-specific integrated circuit (ASIC).

### Equivalents

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method comprising:
(a) obtaining neural activity data for a first subject and a second subject during verbal interaction between the first subject and the second subject; and
b) calculating coherence between the neural activity data for the first subject and the neural activity data for the second subject.

2. The method of claim 1, wherein the neural activity data is functional near-infrared spectroscopy (fNIRS) signals.

3. The method of claim 1, wherein the verbal interaction includes an object naming and narrative task.

4. The method of claim 1, wherein the verbal interaction includes alternating monologues.

5. The method of claim 1, wherein the verbal interaction includes dialogue between the first subject and the second subject.

6. The method of claim 1, wherein coherence is calculated using a cross-correlation algorithm.

7. The method of claim 1, wherein coherence is calculated using a phase- locked wavelet coherence algorithm.

8. The method of claim 1, wherein the first subject is an adult and the second subject is selected from the group consisting of an infant, a toddler, and a child.

9. The method of claim 1, further comprising:
(c) correlating diminished coherence values with affliction with a social disorder.

10. The method of claim 9, wherein the social disorder is a developmental social disorder.

11. The method of claim 9, wherein the social disorder is autism.

12. The method of claim 9, wherein the social disorder is selected from the group consisting of: schizophrenia, post-traumatic stress disorder, and depression.

13. A non-transitory, tangible computer-readable medium comprising computer-readable program instructions for implementing the method of claim 1.

14. A system comprising:
at least two NIRS caps, each of NIRS cap including a plurality of optodes;
an fNIRS system communicatively coupled to the at least two NIRS caps, the fNIRS system programmed to obtain fNIRS data from the at least two NIRS caps when the at least two NIRS caps are worn by subjects; and
a computing device programmed to:
obtain fNIRS data for a first subject and a second subject during verbal interaction between the first subject and the subject; and
calculate coherence between the neural activity data for the first subject and the neural activity data for the second subject.

15. The system of claim 14, wherein the computing device is further programmed to:
correlate diminished coherence values with affliction with a social disorder.

16. The method of claim 1, further comprising:
(d) administering a therapy; and
(e) repeating steps (a)-(d) to assess effectiveness of the therapy.

17. A method comprising:
(a) obtaining functional near-infrared spectroscopy (fNIRS) signals for a first subject and a second subject during verbal interaction between the first subject and the second subject, wherein:
the verbal interaction is selected from the group consisting of: an object naming and narrative task, alternating monologues, and dialogue between the first subject and the second subject;
the first subject is an adult; and
the second subject is selected from the group consisting of an infant, a toddler, and a child;
(b) calculating coherence between the functional near-infrared spectroscopy (fNIRS) signals for the first subject and the functional near-infrared spectroscopy (fNIRS) signals for the second subject using one or more algorithms selected from the group consisting of: a cross-correlation algorithm and a phase-locked wavelet coherence algorithm; and
(c) correlating diminished coherence values with affliction with autism.

18. The method of claim 17, wherein the second subject is the first subject's child.

19. The method of claim 17, further comprising:
(d) administering a therapy; and
(e) repeating steps (a)-(c) to assess effectiveness of the therapy.

20. The method of claim 19, wherein the therapy is early intervention.
